(19) United States
(12) Patent Application Publication
(10) Pub. No.: US 2017/0128417 A1
(43) Pub. Date: May 11, 2017

(54) COMBINATION DRUG THERAPIES FOR CANCER AND METHODS OF MAKING AND USING THEM

(71) Applicant: Vicus Therapeutics, LLC, Morristown, NJ (US)

(72) Inventors: John Maki, Mendham, NJ (US); Newell Bascomb, Hendersonville, NC (US); Fredric Young, Los Altos, CA (US)

(21) Appl. No.: 15/320,733

(22) PCT Filed: Jul. 1, 2015
(86) PCT No.: PCT/US15/38854

§ 371 Date: Dec. 20, 2016

Related U.S. Application Data
(60) Provisional application No. 62/019,821, filed on Jul. 1, 2014.

(51) Int. Cl. A61K 31/407; A61K 45/06; A61K 31/4045; A61K 31/138; A61K 31/155

(52) U.S. Cl. CPC A61K 31/407; A61K 31/138; A61K 31/155; A61K 31/4045; A61K 45/06

(57) ABSTRACT

In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”); a non-steroidal anti-inflammatory drug (a NSAID); and a therapeutic agent, compound or composition comprising: a H₂-receptor antagonist (H₂RA), a melatonin (or an N-acetyl-5-methoxy-tryptamine), a metformin, a quinoline (e.g., chloroquine), an immune checkpoint blockade agent, or an agent that blocks the interaction between a transmembrane programmed cell death 1 protein, or any combination thereof. In alternative embodiments, the therapeutic combinations of therapeutic agents or drugs further comprise an anti-cancer or anti-tumor antibody, a cytokine, and/or a chemotherapeutic agent.

### RELATED APPLICATIONS

This Patent Convention Treaty (PCT) International Application claims the benefit of priority under 35 U.S.C. §119(e) of U.S. Provisional Application Ser. No. (U.S. Ser. No.) 62/019,821, filed Jul. 1, 2014. The aforementioned applications is expressly incorporated herein by reference in its entirety and for all purposes.

### FIELD OF THE INVENTION

This invention relates generally to medicine, pharmaceutical formulations and medical devices. In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”); a non-steroidal anti-inflammatory drug (a NSAID); and a therapeutic agent, compound or composition comprising: a H₂-receptor antagonist (H₂RA), a melatonin (or an N-acetyl-5-methoxytryptamine), a metformin, a quinoline (e.g., chloroquine), an immune checkpoint blockade agent, or an agent that blocks the interaction between a transmembrane programmed cell death 1 protein, or any combination thereof. In alternative embodiments, the therapeutic combinations of therapeutic agents or drugs further comprise an anti-cancer or anti-tumor antibody, a cytokine, and/or a chemotherapeutic agent.

### BACKGROUND

Chemotherapy is important in cancer treatment, but chemotherapy drugs act by damaging high proliferating cells, and damage to normal cells results in chemotherapy toxicities and side effects. Chemotoxicity can be seen most in actively dividing tissues such bone marrow, hair follicles and gastrointestinal mucosa. New approaches in cancer chemotherapeutics are needed to address these challenges.

### SUMMARY

In alternative embodiments, provided are therapeutic combinations of therapeutic agents or drugs for an individual in need thereof comprising or consisting of:

(a)

(i) a beta adrenergic receptor antagonist (a “beta blocker”);

(ii) a non-steroidal anti-inflammatory drug (a NSAID); and

(iii) a therapeutic agent, compound or composition comprising:
(1) a H₂-receptor antagonist (H₂RA),
wherein optionally the H2-receptor antagonist comprises or consists of a cimetidine or equivalent, or TAGAMET™, TAGAMET HB™ or TAGAMET HB200™; a ranitidine or equivalent, or TRITEC™ or ZANTAC™; a famotidine or equivalent, or PEPCIDINE™ or PEPCID™; a nizatidine or equivalent, or TAZAC™ or AXID™,
wherein optionally the H₂-receptor antagonist is dosaged for administration at: (A) for once a day dosing (QD): at 20 mg, 40 mg, or between about 20 to 40 mg; or, (B) for twice a day dosing (BID): at 20 mg, 40 mg, or between about 20 to 40 mg;
(2) a melatonin (also known chemically as N-acetyl-5-methoxytryptamine),
wherein optionally the melatonin is: a recombinant melatonin, or a synthetic melatonin,
wherein optionally the melatonin is dosaged for administration at: for once a day dosing (QD): at 5 mg, 10 mg, 15 mg, 20 mg, or between about 5 to 20 mg;
(3) (i) a metformin, or an N,N-Dimethylimidodicarbonimidic diamide, or a GLUCOPHAGE™, FORTAMET™, GLUMETZA™ or RIOMET™, or a quinoline, an aminoquinoline, e.g., a 4-aminoquinoline or an 8-Aminoquinoline, e.g., a chloroquine (or ARALEN™), a hydroxychloroquine (or PLAQUENIL™) a quinacrine (ATABRINE™), a primaquine, a tafenoquine, or equivalents thereof;
(4) an immune checkpoint blockade agent, or an agent that blocks the interaction between a transmembrane programmed cell death 1 protein (PD-1;

also known as CD279) and its ligand, PD-1 ligand 1 (PD-L1), or an ipilumumab (CTLA-4 mAb) or nivolumab (PD-1 mAb), or a lambrolizumab (a PD-L1 mAb);
(5) an activator of a pattern recognition receptor (PRR) or a toll-like receptor 7 (TLR7), or an imiquimod;
(6) or any combination thereof,
wherein optionally the therapeutic combinations of therapeutic agents or drugs comprises (i), (ii), and a (iii) combination comprising: (1) and (2); (1) and (3); (1) and (4); (1) and (5); (2) and (3), (2) and (4); (2) and (5); (3) and (4); (4) and (5);
r
(b) the therapeutic combination of therapeutic agents or drugs of (a), wherein:
   (i) the non-steroidal anti-inflammatory drug (a NSAID) comprises (a) a cyclooxygenase (COX) (or prostaglandin synthase) inhibitor; or, (b) the COX inhibitor of (a), wherein the COX inhibitor comprises or consists of an etodolac or equivalent; a naproxen or equivalent; a celecoxib or equivalent; a rofecoxib or equivalent; a etoricoxib or equivalent; a valdecoxib or equivalent; a parecoxib or equivalent; a nabumetone or equivalent; a diclofenac (2-(2,6-dichloranilino) phenylacetic acid) or equivalent; or, a lumiracoxib or equivalent; or
   (ii) the neuropathic pain analgesic comprises or consists of gabapentin or pregabalin;
(c) the beta adrenergic receptor antagonist (a beta blocker) comprises propranolol or equivalent,
and optionally the propranolol is INDERAL™, AVLOCARDYL™, DERALIN™, DOCITON™, INDERALICI™, INNOPRAN XL™, or SUMIAL™;
(d) the therapeutic combination of therapeutic agents or drugs of (a), comprising:
   (1) a therapeutic combination of (a)(i), (a)(ii) and (a)(iii)(1),
   (2) a therapeutic combination of (a)(i), (a)(ii) and (a)(iii)(2),
   (3) a therapeutic combination of (a)(i), (a)(ii) and (a)(iii)(3),
   (4) a therapeutic combination of (a)(i), (a)(ii) and (a)(iii)(4),
   (5) a therapeutic combination of (a)(i), (a)(ii) and (a)(iii)(5),
   (6) a therapeutic combination of (a)(i), (a)(ii) and (a)(iii)(6); or
(e) the therapeutic combination of (a)(i) and (a)(ii) comprises or is a VT-122™ (Vicus Therapeutics, Morristown, N.J. ).

In alternative embodiments of the therapeutic combinations of therapeutic agents or drugs, the etodolac is a LODINE™, LODINE SR™ or ECCOXOLAC™; or the celecoxib is CELEBREX™ or CELEBRA™; or the rofecoxib is VIOXX™, CEOXX™ or CEEOXX™; or the etoricoxib is ARCOXIA™, ALGIX™ or TAUXIB™; or the valdecoxib is BEXTRA™; the parecoxib is DYNASTAT™; the naproxen is XENOBID™, ALEVE™, ANAPROX™, MIRANAX™, NAPROGESIC™ NAPROSYN™, NAPRELAN™, PROXEN™ or SYNFLEX™; the nabumetone is RELAFEN™, RELIFEX™ or a GAMBARAN™; or, the diclofenac is FLECTOR

PATCH™, VOLTAREN™, VOLTAROL™, DICLON™, DICLOFLEX DIFEN™ DIFENE™, CATAFLAM™, PENNSAID™, PANAMOR™, RHUMALGAN™, MODIFENAC™, ABITREN™, OLFEN™, VOVERAN™, ARTHROTEC™, DEDOLOR™, DEFLAMAT™, VETAGESIC™ or ZOLTEROL™;

In alternative embodiments, the therapeutic combinations of therapeutic agents or drugs further comprise an anti-cancer or anti-tumor antibody, wherein optionally the anti-cancer or anti-tumor antibody is an alemtuzumab (or CAMPATH™, or LEMTRADA™), a brentuximab vedotin (or ADCETRIS™), a cetuximab (or ERBITUX™), a gemtuzumab ozogamicin (or MYLOTARG™), an ibritumomab tiuxetan (or ZEVALIN™), a nimotuzumab (or TheraCIM™), an ofatumumab (or ARZERRA™), a panitumumab (or VECTIBIX), a rituximab (or RITUXAN™, or ZYTUX™), a tositumomab (or BEXXAR), or a trastuzumab (or HERCLON™, or HERCEPTIN™).

In alternative embodiments, the therapeutic combinations of therapeutic agents or drugs further comprise at least one cytokine, wherein optionally the cytokine comprises an IL-2 or an interferon (IFN), and optionally the interferon is an alpha-IFN or a gamma-IFN; and optionally the IL-2 is a recombinant IL-2, an aldesleukin, or a PROLEUKIN (Prometheus Laboratories), wherein optionally the IL-2, recombinant IL-2, or aldesleukin is dosages at about: 1 to 20, 2 to 10, 4 to 5, or 4.5 millions of IUs per cycle; or is dosaged for: 1 to 5, 2 to 4, or 3 cycles number of cycles of therapy.

In alternative embodiments, the therapeutic combinations of therapeutic agents or drugs further comprise a chemotherapeutic agent,

wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring,

and optionally the inducer of apoptosis or a mitotic inhibitor or anti-microtubule inhibitor comprises or consists of a raltitrexed or equivalent, or TOMUDEX™; a doxorubicin or equivalent, or ADRIAMYCIN™; a fluorouracil or 5-fluorouracil or equivalent; a paclitaxel or equivalent, or TAXOL™ or ABRAXANE™; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or ONCOVIN™; a vinblastin, vinblastine, vindesine, vinflunine, vinorelbine or NAVELBINE™ or equivalent; or, any combination thereof,

and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or ENDOXAN™, CYTOXAN™, NEOSAR™ or REVIMMUNE™; a mechlorethamine or equivalent; a chlormethine or equivalent; a mustine or equivalent; a nitrogen mustard or equivalent; a chlorambucil or equivalent, or LEUKERAN™; or, a combination thereof,

and optionally the topoisomerase inhibitor comprises or consists of an etoposide or equivalent, or EPOSIN™, ETOPOPHOS™, VEPESID™ or VP-16™; an amsacrine or equivalent; a topotecan or equivalent, or HYCAMTIN™; a teniposide or equivalent, or VUMON™ or VM-26™; an epipodophyllotoxin or equivalent; a camptothecin or equivalent; an irinotecan or equivalent, or CAMPTOSAR™; or, a combination thereof,

and optionally the glycopeptide antibiotic comprises or consists of a bleomycin or equivalent or a bleomycin A2 or B2 or equivalent; a mitomycin or a mitomycin C or equivalent, a plicamycin (also known as mithramycin) or equivalent, or MITHRACIN™; or, a combination thereof,

and optionally the steroid receptor inhibitor comprises or consists of an estrogen receptor modulator (a SERM), and optionally the estrogen receptor modulator comprises or consists of a tamoxifen or equivalent, or NOLVADEX™, ISTUBAL™ or VALODEX™, and optionally the steroid inhibitor or an anti-steroid comprises or consists of a finasteride or equivalent, or PROSCAR™, PROPECIA™, FINCAR™, FNPECIA™, FINAX™, FINAST™, FNARA™, FNALO™, PROSTERIDE™, GEFINA™, APPECIA™, FINASTERID IVAX™, FINASTERID or ALTERNOVA™,

and optionally the macrolide or composition comprising a macrolide ring comprises or consists of a clarithromycin or equivalent, or BIAXIN™, KLARICID™, KLABAX™, CLARIPEN™, CLARIDAR™, FROMILID™ or CLACID™; an azithromycin or equivalent, or ZITHROMAX™, ZITROMAX™ or SUMAMED™; a dirithromycin or equivalent; an erythromycin or equivalent; a roxithromycin or equivalent, or ROXO™, SURLID™, RULIDE™, BIAXSIG™, ROXAR™, ROXIMYCIN™ or COROXIN™; a telithromycin or equivalent or KETEK™; a josamycin or equivalent; a kitasamycin or equivalent; a midecamycin or equivalent; oleandomycin or equivalent; a roxithromycin or equivalent, or ROXO™, SURLID™, RULIDE™, BIAXSIG™, ROXAR™, ROXIMYCIN™ or COROXIN™; a troleandomycin or equivalent; or a tylosin or equivalent; or, any combination thereof, wherein optionally the chemotherapeutic agent comprises a sorafenib or equivalent, or NEXAVAR™; a sunitinib or equivalent, or SUTENT™; an erlotinib or equivalent, or TARCEVA™; an imatinib or equivalent, or GLEEVEC™; a lapatinib or equivalent, or TYKERB™; a toceranib or equivalent, or PALLADIA™; a masitinib or equivalent, or MASIVET™, a bevacizumab or equivalent, or AVASTIN™; a trastuzumab or equivalent, or HERCEPTIN™; a cetuximab or equivalent, or ERBITUX™; a bevacizumab or equivalent, or AVASTIN™ or BIBW 2992; a gefitinib or equivalent, or IRESSA™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or TASIGNA™; a panitumumab or equivalent, or VECTIBIX™; a bandetinib or equivalent; a brivanib or equivalent, or E7080™; a thalidomide or equivalent, or THALOMID™; lenalidomide or equivalent, or REVLIMID™; a bortezomib or equivalent, or VELCADE™; disulfiram or equivalent, or ANTABUSE™ or ANTABUS™; or an epigallocatechin gallate (EGCG) or equivalent; a demecolcine, an etoglucid or elsamitrucin, a lonidamine, a lucanthone, a mitotane or a mitoguazone or equivalent; or any combination thereof.

In alternative embodiments, the therapeutic combinations of therapeutic agents or drugs further comprise a radiotherapy enhancing agent.

In alternative embodiments, the therapeutic combinations of therapeutic agents or drugs further comprise a proton pump inhibitor (a PPI), wherein optionally the proton pump inhibitor comprises or consists of a benzimidazole compound or structure, or an imidazopyridine compound or structure.

In alternative embodiments, the therapeutic combinations of therapeutic agents or drugs further comprise a radioactive particle or isotope; or a microscopic, radioactive glass microspheres, or insoluble glass microspheres comprising a yttrium-90, or a THERASPHERE™ (Biocompatibles International, Surry UK); or a drug-storing, drug-carrying or drug-eluting (e.g., a cancer drug-eluting) particle, nanoparticle, liposome or bead, e.g., as a doxorubicin-loaded drug-eluting bead, or a DC Bead®.

In alternative embodiments, the therapeutic combinations of therapeutic agents or drugs further comprise an adjuvant, e.g., any vehicle such as mineral salts, emulsions, liposomes and virosomes; and/or any immunostimulating agent such as Toll-like receptor (TLR) agonists (e.g., monophosphoryl lipid A (MPL)), saponins or cytokines. In alternative embodiments, exemplary adjuvants comprise alum, MPLs, virus-like particles (VLPs) and immunopotentiating reconstituted influenza virosomes (IRIVs), or MF59 or AS03 (GlaxoSmithKline, oil-in-water emulsions).

In alternative embodiments of the therapeutic combinations of therapeutic agents or drugs, two or more drugs of the therapeutic combination are formulated as separate compositions, or two or more drugs of the therapeutic combination are formulated into one composition or drug formulation (two or more drugs of the therapeutic combination are formulated together). In alternative embodiments of the therapeutic combinations of therapeutic agents or drugs, three or more drugs of the therapeutic combination are formulated as separate compositions, or three or more drugs of the therapeutic combination are formulated into one composition or drug formulation (three or more drugs of the therapeutic combination are formulated together).

In alternative embodiments of the therapeutic combinations of therapeutic agents or drugs, the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are formulated in different compositions or formulations, or, are formulated in the same composition or formulation, or are formulated together.

In alternative embodiments of the therapeutic combinations of therapeutic agents or drugs, one or two or more or all of the drugs of the therapeutic combination are packaged individually, or are packaged together, or packaged in any combination, in a single package, a plurality of packages or packettes, or a blister packet, lidded blister or blister card or packets, or a shrink wrap.

In alternative embodiments of the therapeutic combinations of therapeutic agents or drugs, the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are packaged individually in a single package, a plurality of packages or packettes, or a blister packet, lidded blister or blister card or packets, or a shrink wrap. In alternative embodiments, one or two or more or all of the drugs of the therapeutic combination are packaged together or in any combination in a single package, a plurality of packages or packettes, or a blister packet, lidded blister or blister card or packets, or a shrink wrap. In alternative embodiments, two or more or all of the drugs are released upon opening of the single package, plurality of packages or packettes, blister packet, lidded blister, blister card or packets or shrink wrap. In alternative embodiments, the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer are packaged together in a single package, a plurality of packages or packettes, or a blister packet, lidded blister or blister card or packets, or a shrink wrap, and two or more or all of the drugs are released upon opening of the single package, plurality of packages or packettes, blister packet, lidded blister, blister card or packets or shrink wrap.

In alternative embodiments, one, two or three or more or all of the drugs of the therapeutic combination are formulated or manufactured as a parenteral formulation, an aqueous solution, a liposome, an injectable solution, a tablet, a pill, a lozenge, a capsule, a caplet, a patch, a spray, an inhalant, a powder, a freeze-dried powder, an inhalant, a patch, a gel, a geltab, a nanosuspension, a nanoparticle, a nanoliposome, a microgel, a pellet, a suppository or any combination thereof.

In alternative embodiments, one, two or three or more or all of the drugs of the therapeutic combination are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one nanosuspension, one nanoparticle, one nanoliposome, one microgel or one suppository.

In alternative embodiments: (a) the dosage of etodolac ranges from about 200 mg to 400 mg a day, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more; or, (b) the dosage of propranolol ranges from 10 to 320 mg per day based on heart rate and blood pressure of the individual, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more.

In alternative embodiments of the therapeutic combination, the drug combination is packaged in dosages that match a chrono-dosing regimen to match an optimal dose for the time of day. For example, in exemplary alternative embodiments, the beta adrenergic receptor antagonist or a beta blocker or equivalent, or a propranolol or equivalent; the non-steroidal anti-inflammatory drug or NSAID or equivalent, or etodolac or equivalent; and the therapeutic agent for the treatment of cancer, are packaged in dosages that match a chrono-dosing regimen to match an optimal dose for the time of day.

In exemplary alternative embodiments, the beta adrenergic receptor antagonist or beta blocker or equivalent or propranolol or equivalent; the non-steroidal anti-inflammatory drug or NSAID or equivalent or etodolac or equivalent; and the therapeutic agent for the treatment of cancer, are packaged in dosages that match a chrono-dosing regimen comprising:

(a) in the AM, 20 mg beta adrenergic receptor antagonist (a beta blocker), e.g., a propranolol or equivalent, 200 mg NSAID, e.g., an etodolac or equivalent; in the afternoon, 10 mg beta blocker, 200 mg NSAID, e.g., an etodolac or equivalent; in the PM, 10 mg beta blocker, 400 mg NSAID;

(b) in the AM 40 mg beta adrenergic receptor antagonist (a beta blocker), e.g., a propranolol or equivalent, 200 mg NSAID, e.g., an etodolac or equivalent; in the afternoon 20 mg beta blocker, 200 mg NSAID; in the evening, 20 mg propranolol, 400 mg NSAID;

(c) in the AM 80 mg beta adrenergic receptor antagonist (a beta blocker), e.g., a propranolol or equivalent, 200 mg NSAID; in the afternoon 40 mg beta blocker, 200 mg NSAID, in the evening 40 mg, NSAID; or

(d) a dose escalation comprising a regimen of (a) to (b) to (c).

In exemplary alternative embodiments, the beta adrenergic receptor antagonist or beta blocker or equivalent or propranolol or equivalent; the non-steroidal anti-inflammatory drug or NSAID or equivalent or etodolac or equivalent; and the therapeutic agent for the treatment of cancer, are packaged in dosages that match a chrono-dosing regimen comprising:

Start: AM, 20 mg propranolol, 200 mg etodolac; afternoon, 10 mg propranolol, 200 mg etodolac; PM 5 mg propranolol, 400 mg etodolac;

Dose Escalation 1: AM 40 mg propranolol, 200 mg etodolac; afternoon 20 mg propranolol, 200 mg etodolac; evening, 10 mg propranolol, 400 mg etodolac;

Dose escalation 2: AM 80 mg propranolol, 200 mg etodolac; afternoon 40 mg propranolol, 200 mg etodolac, evening 20 mg, etodolac.

In exemplary alternative embodiments, the therapeutic drug combination is formulated for administration once a day, b.i.d. or t.i.d, or weekly, or biweekly, or monthly. In exemplary alternative embodiments, the beta adrenergic receptor antagonist (a beta blocker) or propranolol or equivalent; the non-steroidal anti-inflammatory drug or NSAID or etodolac or equivalent; and the therapeutic agent for the treatment of cancer, are formulated for administration once a day, b.i.d. or t.i.d, or weekly, or biweekly, or monthly.

In exemplary alternative embodiments, the therapeutic combination of drugs are formulated for administration intravenously, topically, orally, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings.

In alternative embodiments, provided are a device, a medical device, an implant, a breast implant, a prosthesis, a stent, a catheter, comprising a therapeutic combination of therapeutic agents or drugs as provided herein.

In alternative embodiments, provided are a pharmaceutical composition or formulation comprising the therapeutic combination as provided herein, and optionally the pharmaceutical composition or formulation can further comprise a pharmaceutically acceptable excipient. In exemplary alternative embodiments, the pharmaceutical composition or formulation is formulated or manufactured as a feed, a food, a food or feed concentrate, a pellet, a lozenge, a liquid, a lotion, an implant, a nanoparticle, an elixir, an aerosol, a spray, an aerosol, an inhalant, a powder, a tablet, a pill, a capsule, a gel, a geltab, a nanosuspension, a nanoparticle, a patch, a microgel or a suppository.

In alternative embodiments, provided are methods for treating, preventing or ameliorating a tumor or a cancer, comprising: applying or administering to an individual in need thereof; or, applying or administering to an effected tissue: the therapeutic combinations as provided herein, or a pharmaceutical composition or formulation as provided herein,

wherein optionally the therapeutic agents or drugs are administered separately or together, or at the same time, or in synchrony, or by chrono-dosing, or one of the therapeutic agents or drugs is administered before another of the therapeutic agents or drugs, and optionally the therapeutic agents or drugs are formulated for administration intravenously (IV), parenterally, nasally, topically or locally, orally, or by liposome, implant or vessel-targeted nanoparticle delivery.

In exemplary alternative embodiments of the methods, the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

In alternative embodiments, provided are methods for treating, preventing or ameliorating a tumor or a cancer, comprising:

(a) applying or administering to an individual in need thereof; or, applying or administering to an effected tissue; the therapeutic combination as provided herein, or a pharmaceutical composition or formulation as provided herein,

wherein optionally the therapeutic agents or drugs are administered separately or together, or at the same time, or in synchrony, or by chrono-dosing, or one of the therapeutic agents or drugs is administered before another of the therapeutic agents or drugs,

and optionally the therapeutic agents or drugs are formulated for administration intravenously (IV), parenterally, nasally, topically or locally, orally, or by liposome, implant or vessel-targeted nanoparticle delivery; and

(b) administering to the individual in need thereof:
(i) a systemic anti-cancer or anti-tumor treatment, wherein optionally the systemic anti-cancer or anti-tumor treatment comprises administration of a drug, a biologic, a nutrient, an anti-cancer or anti-tumor dietary regimen, a radioactive agent, a tumor ablative agent, or
(ii) an anti-cancer or anti-tumor radiotherapy or a proton beam therapy,

wherein the therapeutic combination or pharmaceutical composition or formulation of (a) is administered before the anti-cancer or anti-tumor treatment of (b), or both are administered consecutively, or the therapeutic combination or pharmaceutical composition or formulation of (a) is administered after the anti-cancer or anti-tumor treatment of (b), or any combination thereof.

In exemplary alternative embodiments the methods further comprising: an anti-cancer or anti-tumor radiotherapy or a proton beam therapy.

In alternative embodiments, provided are uses of a therapeutic combination as provided herein in the manufacture of a medicament. In alternative embodiments, provided are uses of the therapeutic combinations as provided herein, in the manufacture of a medicament for treating a cancer or a tumor. In exemplary alternative embodiments the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a Histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer.

The details of one or more aspects of the invention are set forth in the description below. Other features, objects, and advantages of the invention will be apparent from the description and from the claims.

All publications, patents and patent applications cited herein are hereby expressly incorporated by reference for all purposes.

### DETAILED DESCRIPTION

In alternative embodiments, provided are therapeutic combinations, pharmaceutical compositions, formulations, kits and devices for treating, preventing or ameliorating a tumor or a cancer, and methods for treating, preventing or ameliorating a tumor or a cancer. In alternative embodiments, provided are therapeutic combinations, pharmaceutical compositions, formulations, kits and devices comprising: a beta adrenergic receptor antagonist (a “beta blocker”); a non-steroidal anti-inflammatory drug (a NSAID); and a therapeutic agent, compound or composition comprising: a H₂-receptor antagonist (H₂RA), a melatonin (or an N-acetyl-5-methoxytryptamine), a metformin, a quinoline (e.g., chloroquine), an immune checkpoint blockade agent, or an agent that blocks the interaction between a transmembrane programmed cell death 1 protein, or any combination thereof.

In alternative embodiments the cancer is a dysfunctional cell condition. In alternative embodiments the cancer or dysfunctional cell condition comprises (is) any metastatic or benign tumor, and the methods or uses as provided herein are used for ameliorating, treating (killing, eliminating, stopping the growth and/or metastasis of) cancer stem cells or cancer cells from: lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, a neoplasm of the central nervous system (CNS), primary CNS lymphoma, spinal axis tumors, brain stem glioma or pituitary adenoma, and any combination thereof.

In alternative embodiments provided are products of manufacture comprising a blister package, a lidded blister or a blister card or packet, a clamshell, a tray or a shrink wrap comprising a therapeutic combination as provided herein, or the pharmaceutical composition or formulation as provided herein. In alternative embodiments the products of manufacture can comprise a blister package, a lidded blister or a blister card or packet, a clamshell, a tray or a shrink wrap comprising a therapeutic combination as provided herein, or the pharmaceutical composition or formulation as provided herein, wherein the therapeutic combination or pharmaceutical composition or formulation are manufactured and/or formulated for at least two, three, four or five or more dosage administrations; or the therapeutic combination or pharmaceutical composition or formulation are manufactured and/or formulated for once a day, or bid (twice a day), or tid (three times a day), or four times a day, administration.

In alternative embodiments, a drug combination as provided herein is formulated, packaged or designed for drug regimen compliance of a cancer patient population, a pediatric or geriatric population, or a mentally compromised patient population.

In alternative embodiments drug combination(s) as provided herein are formulated, packaged or designed for drug regimen compliance of a cancer patient population having mild or severe mental retardation, slow cognition, dementia, senility, Alzheimer's disease, traumatic brain injury, chemical brain damage, mental diseases (e.g., dissociative disorder, obsessive-compulsive disorder, delusional disorder, schizophrenia, mania, panic disorder, depression, dyslexia, any learning disability and the like) post-traumatic stress disorder, traumatic war neurosis, post-traumatic stress syndrome (PTSS), physical disability (e.g., blindness).

In alternative embodiments of the products of manufacture as provided herein the therapeutic combination or pharmaceutical composition or formulation are formulated (e.g., manufactured) as one dosage administration in the morning and one dosage administration in the evening; or are formulated as one dosage administration in the morning, one dosage mid-day and one dosage administration in the evening. In one aspect, the dosage schedule provides a relatively higher dose of one drug in the morning (the AM) than in the evening, and a relatively higher dose of another medication in the evening than in the morning. For example, in alternative embodiments the therapeutic combination or the pharmaceutical composition are formulated for multiple administrations, e.g., at least two administrations, one in the morning and one in the evening, wherein the dosage schedule provides a relatively higher dose of beta blocker in the morning (the AM) than in the evening, and a relatively higher dose of an anti-inflammatory medication in the evening than in the morning.

In alternative embodiments, the products of manufacture or formulations as provided herein comprise a therapeutic combination as provided herein or the pharmaceutical composition or formulation as provided herein, and a nutritional supplement, or food supplement or feed supplement.

### Methods of Administration

In alternative embodiments, provided herein are therapeutic combinations of drugs, pharmaceutical compositions, preparations and kits, that can be administered by several routes, for formulated for administration by any of several routes, including intravenous, topical and oral, or combinations thereof

For example, one embodiment comprises a product of manufacture comprising a pharmaceutical composition or a formulation, a blister package, a lidded blister or a blister card or packet, a clamshell, a tray or a shrink wrap, or a kit, comprising: therapeutic combinations of drugs, pharmaceutical compositions or preparations as provided herein.

In alternative embodiments, although all ingredients can be in one blister package, a lidded blister or a blister card or packet, a clamshell, a tray or a shrink wrap, or a kit, separate ingredients can be formulated e.g., for topical application, for oral or for topical application. Each ingredient can be either separately packaged, or can be formulated as one unit dose, e.g., as one tube (e.g., with gel, lotion etc.), ampoule, blister packette and the like.

In alternative embodiments, provided herein are forms of compositions, preparations and kits that can be administered by inhalation, infusion or injection, (e.g., intraperitoneal, intramuscular, subcutaneous, intra-aural, intra-articular, intra-mammary, etc.), topical application (e.g., on areas, such as eyes, ears, skin or on afflictions such as wounds, burns, etc.), and by absorption through epithelial or mucocutaneous linings (e.g.

vaginal and other epithelial linings, gastrointestinal mucosa, etc.). Methods are known for making compositions, preparations and kits containing the present components that are suitable for each of these methods of administration as well as other methods of administration that are known in the art.

In alternative embodiments, provided herein are compositions, preparations and kits in liquid forms that can be administered orally. The compositions, preparations and kits can be also prepared as capsules, gels, geltabs, tablets, powders, sprays, aerosols, pellets (e.g. for animal consumption), suppositories, lotions, patches or adhesives (e.g., for the skin), or creams and ointments. The compositions, preparations and kits can be also prepared as physiological solutions suitable for I.V. administration or other parenteral administration.

In one aspect, a multi-ingredient kit as provided herein comprises (contains) two or more ingredients. An amount may be determined, e.g. by mass or by weight or by molar amount. In another aspect, a multi-ingredient kit may contain two or more ingredients in unequal amounts. In another aspect, a multi-ingredient kit may contain two or more ingredients in approximately equal amounts and/or one or more ingredients that are not in unequal amounts.

In another embodiment, said multi-ingredient kit may contain two or more ingredients that are admixed. In another aspect, said multi-ingredient kit may contain two or more ingredients that are not admixed. In another aspect, said multi-ingredient kit may contain two or more ingredients that are partially admixed. In another aspect, said multi-ingredient kit may contain two or more ingredients that are at least partially admixed, as well as one or more ingredients that are not admixed. An ingredient in a multi-ingredient kit may be liquid forms that can be administered orally.

### Packaging

In alternative embodiments, provided are therapeutic combinations, preparations, formulations and/or kits, comprising combinations of ingredients, as described herein. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package is made up of two separate elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, provided are blister packs, clamshells or trays comprising a composition (e.g., a (the multi-ingredient combination of drugs as provided herein) combination of active ingredients) as provided herein. Blister packs, clamshells or trays can be designed to be non-reclosable, so consumers can tell if a package has already opened. They are used to package for sale goods where product tampering is a consideration, such as the pharmaceuticals as provided herein. In one aspect, a blister pack as provided herein comprises a molded PVC base, with raised areas (the “blisters”) to contain the tablets, pills, etc. comprising the combinations as provided herein, covered by a foil laminate. Tablets, pills, etc. are removed from the pack either by peeling the foil back or by pushing the blister to force the tablet to break the foil. In one aspect, a specialized form of a blister pack is a strip pack. In one aspect, in the United Kingdom, blister packs adhere to British Standard 8404.

In one aspect, a blister packs also comprise a method of packaging where the compositions comprising combinations of ingredients as provided herein are contained inbetween a card and a clear PVC. The PVC can be transparent so the item (pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed pills, tablets, geltabs, etc., the card has a perforated window for access. In one aspect, more secure blister packs, e.g., for items such as pills, tablets, geltabs, etc. as provided herein are used, and they can comprise of two vacuum-formed PVC sheets meshed together at the edges, with the informative card inside. These can be hard to open by hand, so a pair of scissors or a sharp knife may be required to open.

In one aspect, blister packaging comprises at least two components (e.g., is a multi-ingredient combination of drugs as provided herein): a thermoformed “blister” which houses the product (e.g., a pharmaceutical combination as provided herein), and then a “blister card” that is a printed card with an adhesive coating on the front surface. During the assembly process, the blister component, which is most commonly made out of PVC, is attached to the blister card using a blister machine. This machine introduces heat to the flange area of the blister which activates the glue on the card in that specific area and ultimately secures the PVG blister to the printed blister card. The thermoformed PVG blister and the printed blister card can be as small or as large as you would like, but there are limitations and cost considerations in going to an oversized blister card. Conventional blister packs can also be sealed (e.g., using an AERGO 8 DUO™, SCA Consumer Packaging, Inc., DeKalb Ill.) using regular heat seal tooling. This alternative aspect, using heat seal tooling, can seal common types of thermoformed packaging.

### Blister Packaging

In alternative embodiments, provided are therapeutic combinations, preparations, formulations and/or kits can be manufactured as “blister packages” or as a plurality of packettes, including as lidded blister packages, lidded blister or blister card or packets or packettes, or a shrink wrap.

In alternative embodiments, laminated aluminum foil blister packs are used, e.g., for the preparation of drugs designed to dissolve immediately in the mouth of a patient. This exemplary process comprises having the drug combinations as provided herein prepared as an aqueous solution(s) which are dispensed (e.g., by measured dose) into an aluminum (e.g., alufoil) laminated tray portion of a blister pack. This tray is then freeze-dried to form tablets which take the shape of the blister pockets. The alufoil laminate of both the tray and lid fully protects any highly hygroscopic and/or sensitive individual doses. In one aspect, the pack incorporates a child-proof peel open security laminate. In one aspect, the system give tablets an identification mark by embossing a design into the alufoil pocket that is taken up by the tablets when they change from aqueous to solid state. In one aspect, individual ‘push-through’ blister packs/ packettes are used, e.g., using hard temper aluminum (e.g., alufoil) lidding material. In one aspect, hermetically-sealed high barrier aluminum (e.g., alufoil) laminates are used. In one aspect, any products of manufacture as provided herein, including kits or blister packs, use foil laminations and strip packs, stick packs, sachets and pouches, peelable and non-peelable laminations combining foil, paper, and film for high barrier packaging.

In alternative embodiments, any products of manufacture as provided herein, including kits or blister packs, include memory aids to help remind patients when and how to take the drug. This safeguards the drug's efficacy by protecting each pill until it's taken; gives the product or kit portability, makes it easy to take a dose anytime or anywhere.

A number of aspects of the invention have been described. Nevertheless, it will be understood that various modifications may be made without departing from the spirit and scope of the invention. Accordingly, other aspects are within the scope of the following claims.

1. A therapeutic combination of therapeutic agents or drugs for an individual in need thereof comprising or consisting of:
(a)
(i) a beta adrenergic receptor antagonist (a “beta blocker”);
(ii) a non-steroidal anti-inflammatory drug (a NSAID); and
(iii) a therapeutic agent, compound or composition comprising:
(1) a H₂-receptor antagonist (H₂RA),
wherein optionally the H₂-receptor antagonist comprises or consists of a cimetidine or equivalent, or Tagamet™, Tagamet HB™ or Tagamet HB200™; a ranitidine or equivalent, or TRITEC™ or ZANTAC™; a famotidine or equivalent, or Pepcidine™ or Pepcid™; a nizatidine or equivalent, or TAZAC™ or AXID™.
wherein optionally the H₂-receptor antagonist is dosaged for administration at: (A) for once a day dosing (QD): at 20 mg, 40 mg, or between about 20 to 40 mg; or, (B) for twice a day dosing (BID): at 20 mg, 40 mg, or between about 20 to 40 mg;
(2) a melatonin (also known chemically as N-acetyl-5-methoxytryptamine),
wherein optionally the melatonin is: a recombinant melatonin, or a synthetic melatonin,
wherein optionally the melatonin is dosaged for administration at: for once a day dosing (QD): at 5 mg, 10 mg, 15 mg, 20 mg, or between about 5 to 20 mg;
(3) a metformin, or an N,N-Dimethylimidodicarbonimidic diamide, or a Glucophage™, Fortamet™, Glumetza™ or Riomet™, or a quinoline, an aminoquinoline, e.g., a 4-aminoquinoline or an 8-Aminoquinoline, e.g., a chloroquine (or Aralen™), a hydroxychloroquine (or Plaquenil™) a quinacrine (Atabrine™), a primaquine, a tafenoquine, or equivalents thereof;
(4) an immune checkpoint blockade agent, or an agent that blocks the interaction between a transmembrane programmed cell death 1 protein (PD-1; also known as CD279) and its ligand, PD-1 ligand 1 (PD-L1), or an ipilumumab (CTLA-4 mAb) or nivolumab (PD-1 mAb), or a lambrolizumab (a PD-L1 mAb);
(5) an activator of a pattern recognition receptor (PRR) or a toll-like receptor 7 (TLR7), or an imiquimod;
(6) or any combination thereof; or
(b) the therapeutic combination of therapeutic agents or drugs of (a), wherein:
(i) the non-steroidal anti-inflammatory drug (a NSAID) comprises (a) a cyclooxygenase (COX) (or prostaglandin synthase) inhibitor; or, (b) the COX inhibitor of (a), wherein the COX inhibitor comprises or consists of an etodolac or equivalent; a naproxen or equivalent; a celecoxib or equivalent; a rofecoxib or equivalent; a etoricoxib or equivalent; a valdecoxib or equivalent; a parecoxib or equivalent; a nabumetone or equivalent; a diclofenac (2-(2,6-dichloranilino) phenylacetic acid) or equivalent; or, a lumiracoxib or equivalent; or
(ii) the neuropathic pain analgesic comprises or consists of gabapentin or pregabalin;
(c) the beta adrenergic receptor antagonist (a beta blocker) comprises propranolol or equivalent,
and optionally the propranolol is INDERAL™, Avlocardyl™, Deralin™, Dociton™, Inderalici™, InnoPran XL™, or Sumial™;
(d) the therapeutic combination of therapeutic agents or drugs of (a), comprising:
(1) a therapeutic combination of (a)(i), (a)(ii) and (a)(iii)(1),
(2) a therapeutic combination of (a)(i), (a)(ii) and (a)(iii)(2),
(3) a therapeutic combination of (a)(i), (a)(ii) and (a)(iii)(3),
(4) a therapeutic combination of (a)(i), (a)(ii) and (a)(iii)(4),
(5) a therapeutic combination of (a)(i), (a)(ii) and (a)(iii)(5),
(6) a therapeutic combination of (a)(i), (a)(ii) and (a)(iii)(6); or
(e) the therapeutic combination of (a)(i) and (a)(ii) comprises or is a VT-122™ (Vicus Therapeutics, Morristown, N.J.).

2. The therapeutic combination of therapeutic agents or drugs of claim 1, wherein the etodolac is a LODINE™, LODINE SR™ or eccoxolac™; or the celecoxib is Celebrex™ or Celebra™; or the rofecoxib is Vioxx™, Ceoxx™ or Ceeoxx™; or the etoricoxib is Arcoxia™, Algix™ or Tauxib™; or the valdecoxib is BEXTRA™; the parecoxib is Dynastat™; the naproxen is Xenobid™, Aleve™, Anaprox™, Miranax™, Naprogesic™, Naprosyn™, Naprelan™, Proxen™ or Synflex™; the nabumetone is Relafen™, Relifex™ or a Gambaran™; or, the diclofenac is Flector patch™, Voltaren™, Voltarol™, Diclon™, Dicloflex Difen™, Difene™, Cataflam™, Pennsaid™, Panamor™, Rhumalgan™, Modifenac™, Abitren™, Olfen™, Voveran™, Arthrotec™, Dedolor™, Deflamat™, Vetagesic™, or Zolterol™.

3. The therapeutic combination of therapeutic agents or drugs of claim 1, further comprising an anti-cancer or anti-tumor antibody,
wherein optionally the anti-cancer or anti-tumor antibody is an alemtuzumab, a brentuximab vedotin, a cetuximab, a gemtuzumab ozogamicin, an abritumomab tiuxetan, a nimotuzumab, an ofatumumab, a panitumumab, a rituximab, a tositumomab, or a trastuzumab.

4. The therapeutic combination of therapeutic agents or drugs of claim 1, further comprising:
(a) a cytokine,
wherein optionally the cytokine comprises an IL-2 or an interferon (IFN),
and optionally the interferon is an alpha-IFN or a gamma-IFN;
and optionally the IL-2 is a recombinant IL-2, an aldesleukin, or a Proleukin (Prometheus Laboratories),
wherein optionally the IL-2, recombinant IL-2, or aldesleukin is dosages at about: 1 to 20, 2 to 10, 4 to 5, or 4.5 millions of IUs per cycle; or is dosaged for: 1 to 5, 2 to 4, or 3 cycles number of cycles of therapy
(b) a chemotherapeutic agent,
wherein optionally the chemotherapeutic agent comprises a doxorubicin or a carboplatin, or comprises an inducer of apoptosis or a mitotic inhibitor or anti-microtubule inhibitor, or an alkylating agent, or a topoisomerase inhibitor, or a glycopeptide antibiotic, or steroid receptor inhibitor, or a matrix metalloproteinase (MMP) inhibitor, or an mTOR (mammalian target of rapamycin) inhibitor, or a macrolide or a composition comprising a macrolide ring,
and optionally the inducer of apoptosis or a mitotic inhibitor or anti-microtubule inhibitor comprises or consists of a raltitrexed or equivalent, or Tomudex™; a doxorubicin or equivalent, or ADRIAMYCIN™; a fluorouracil or 5-fluorouracil or equivalent; a paclitaxel or equivalent, or TAXOL™ or ABRAXANE™; a docetaxel or equivalent, or TAXOTERE™; a larotaxel, tesetaxel or ortataxel or equivalent; an epothilone or an epothilone A, B, C, D, E or F or equivalent; an ixabepilone (also known as azaepothilone B) or equivalent, or BMS-247550™; a vincristine (also known as leurocristine) or equivalent, or Oncovin™; a vinblastin, vinblastine, vindesine, vinflunine, vinorelbine or Navelbine™ or equivalent; or, any combination thereof,
and optionally the alkylating agent comprises or consists of a cisplatin or equivalent; a cisplatinum or equivalent; a cis-diamminedichloridoplatinum(II) (CDDP) or equivalent; a carboplatin or equivalent; a oxaloplatin or equivalent; a cyclophosphamide (cytophosphane) or equivalent, or Endoxan™, Cytoxan™, Neosar™ or Revimmune™; a mechlorethamine or equivalent; a chlormethine or equivalent; a mustine or equivalent; a nitrogen mustard or equivalent; a chlorambucil or equivalent, or Leukeran™; or, a combination thereof,
and optionally the topoisomerase inhibitor comprises or consists of an etoposide or equivalent, or Eposin™, Etopophos™, Vepesid™ or VP-16™; an amsacrine or equivalent; a topotecan or equivalent, or Hycamtin™ a teniposide or equivalent, or Vumon™ or VM-26™; an epipodophyllotoxin or equivalent; a camptothecin or equivalent; an irinotecan or equivalent, or Camptosar™; or, a combination thereof,
and optionally the glycopeptide antibiotic comprises or consists of a bleomycin or equivalent or a bleomycin A₂ or B₂ or equivalent; a mitomycin or a mitomycin C or equivalent, a plicamycin (also known as mithramycin) or equivalent, or Mithracin™; or, a combination thereof,
and optionally the steroid receptor inhibitor comprises or consists of an estrogen receptor modulator (a SERM), and optionally the estrogen receptor modulator comprises or consists of a tamoxifen or equivalent, or Nolvadex™, Istubal™ or Valodex™, and optionally the steroid inhibitor or an anti-steroid comprises or consists of a finasteride or equivalent, or Proscar™, Propecia™, Fincar™, Finpecia™, Finax™, Finast™, Finara™, Finalo™, Prosteride™, Gefina™, Appecia™, Finasterid IVAX™, Finasterid or Alternova™,
and optionally the macrolide or composition comprising a macrolide ring comprises or consists of a clarithromycin or equivalent, or Biaxin™, Klaricid™, Klabax™, Claripen™, Claridar™, Fromilid™ or Clacid™; an azithromycin or equivalent, or ZITHROMAX™, Zitromax™ or Sumamed™; a dirithromycin or equivalent; an erythromycin or equivalent; a roxithromycin or equivalent, or Roxo™, Surlid™, Rulide™, Biaxsig™, ROXar™, Roximycin™ or Coroxin™; a telithromycin or equivalent or KETEK™; a josamycin or equivalent; a kitasamycin or equivalent; a midecamycin or equivalent; oleandomycin or equivalent; a roxithromycin or equivalent, or Roxo™, Surlid™, Rulide™, Biaxsig™, ROXar™, Roximycin™ or Coroxin™; a troleandomycin or equivalent; or a tylosin or equivalent; or, any combination thereof,
wherein optionally the chemotherapeutic agent comprises a sorafenib or equivalent, or Nexavar™; a sunitinib or equivalent, or SUTENT™; an erlotinib or equivalent, or Tarceva™; an imatinib or equivalent, or GLEEVEC™; a lapatinib or equivalent, or Tykerb™; a toceranib or equivalent, or Palladia™; a masitinib or equivalent, or MASIVET™, a bevacizumab or equivalent, or Avastin™; a trastuzumab or equivalent, or HERCEPTIN™; a cetuximab or equivalent, or Erbitux™; a bevacizumab or equivalent, or Avastin™ or BIBW 2992; a gefitinib or equivalent, or Iressa™; a ranibizumab or equivalent, or LUCENTIS™; a pegaptanib or equivalent, or MACUGEN™; a dasatinib or equivalent, or BMS-354825™; a sunitinib or equivalent, or SUTENT™; a pazopanib or equivalent; a nilotinib or equivalent, or Tasigna™; a panitumumab or equivalent, or Vectibix™; a bandetinib or equivalent; a brivanib or equivalent, or E7080™; a thalidomide or equivalent, or THALOMID™; lenalidomide or equivalent, or Revlim™; a bortezomib or equivalent, or VELCADE™; disulfiram or equivalent, or Antabuse™ or Antabus™; or an epigallocatechin gallate (EGCG) or equivalent; a demecolcine, an etoglucid or elsamitrucin, a lonidamine, a lucanthone, a mitotane or a mitoguazone or equivalent; or any combination thereof;
(c) a radiotherapy enhancing agent;
(d) a proton pump inhibitor (a PPI),
wherein optionally the proton pump inhibitor comprises or consists of a benzimidazole compound or structure, or an imidazopyridine compound or structure;
(e) a radioactive particle or isotope; or a microscopic, radioactive glass microsphere, or a TheraSphere; or a drug-eluting or a cancer drug-eluting particle, liposome or bead, or a doxorubicin-loaded drug-eluting bead, or a DC Bead®;
(f) an adjuvant; or
(g) any combination of (a) to (f).

5-9. (canceled)

10. The therapeutic combination of claim 1, wherein two or more drugs of the therapeutic combination are formulated as separate compositions, or two or more drugs of the therapeutic combination are formulated into one composition or drug formulation, or two or more drugs of the therapeutic combination are formulated together.

11. The therapeutic combination of claim 10, wherein the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are formulated in different compositions or formulations, or, are formulated in the same composition or formulation, or are formulated together.

12. The therapeutic combination combination of claim 1, wherein one or two or more or all of the drugs of the therapeutic combination are packaged individually, or are packaged together, or packaged in any combination, in a single package, a plurality of packages or packettes, or a blister packet, lidded blister or blister card or packets, or a shrink wrap.

13. The therapeutic combination combination of 1, wherein the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer, are packaged individually in a single package, a plurality of packages or packettes, or a blister packet, lidded blister or blister card or packets, or a shrink wrap.

14. The therapeutic combination combination of claim 1, wherein one or two or more or all of the drugs of the therapeutic combination are packaged together or in any combination in a single package, a plurality of packages or packettes, or a blister packet, lidded blister or blister card or packets, or a shrink wrap.

15. The therapeutic combination of claim 14, wherein two or more or all of the drugs are released upon opening of the single package, plurality of packages or packettes, blister packet, lidded blister, blister card or packets or shrink wrap.

16. The therapeutic combination combination of claim 1, wherein the beta adrenergic receptor antagonist, or a beta blocker or equivalent, or a propranolol or equivalent; the non-steroidal anti-inflammatory drug, or a NSAID or equivalent, or an etodolac or equivalent; and the therapeutic agent for the treatment of cancer are packaged together in a single package, a plurality of packages or packettes, or a blister packet, lidded blister or blister card or packets, or a shrink wrap, and two or more or all of the drugs are released upon opening of the single package, plurality of packages or packettes, blister packet, lidded blister, blister card or packets or shrink wrap.

17. The therapeutic combination combination of claim 1, wherein one or two or more or all of the drugs of the therapeutic combination are formulated or manufactured as a parenteral formulation, an aqueous solution, a liposome, an injectable solution, a tablet, a pill, a lozenge, a capsule, a caplet, a patch, a spray, an inhalant, a powder, a freeze-dried powder, an inhalant, a patch, a gel, a geltab, a nanosuspension, a nanoparticle, a nanoliposome, a microgel, a pellet, a suppository or any combination thereof; or,
the therapeutic combination of drugs are formulated for administration intravenously, topically, orally, by inhalation, by infusion, by injection, by inhalation, intraperitoneally, intramuscularly, subcutaneously, intra-aurally, for intra-articular administration, for intra-mammary administration, for topical administration or for absorption through epithelial or mucocutaneous linings; or
one or two or more or all of the drugs of the therapeutic combination are formulated or manufactured together in one parenteral formulation, one aqueous solution, one liposome, one injectable solution, one freeze-dried powder, one feed, one food, one food supplement, one pellet, one lozenge, one liquid, one elixir, one aerosol, one inhalant, one adhesive, one spray, one powder, one freeze-dried powder, one patch, one tablet, one pill, one capsule, one gel, one geltab, one lozenge, one caplet, one nanosuspension, one nanoparticle, one nanoliposome, one microgel or one suppository.

18. (canceled)

19. The therapeutic combination combination of claim 1, wherein:
(a) the dosage of etodolac ranges from about 200 mg to 400 mg a day, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more; or
(b) the dosage of propranolol ranges from 10 to 320 mg per day based on heart rate and blood pressure of the individual, or, about 10, 15, 20, 25, 30, 35, 40, 45, 50, 75, 80, 85, 90, 100, 150, 200, 250, 300, 350, 400, 450, 500, 600, 700, 800, 900 or 1000 mg or more.

20. The therapeutic combination combination of claim 1, the drug combination is packaged in dosages that match a chrono-dosing regimen to match an optimal dose for the time of day, or the beta adrenergic receptor antagonist or a beta blocker or equivalent, or a propranolol or equivalent; the non-steroidal anti-inflammatory drug or NSAID or equivalent, or etodolac or equivalent; and the therapeutic agent for the treatment of cancer, are packaged in dosages that match a chrono-dosing regimen to match an optimal dose for the time of day.

21. (canceled)

22. The therapeutic combination combination of claim 1, wherein the beta adrenergic receptor antagonist or beta blocker or equivalent or propranolol or equivalent; the non-steroidal anti-inflammatory drug or NSAID or equivalent or etodolac or equivalent; and the therapeutic agent for the treatment of cancer, are packaged in dosages that match a chrono-dosing regimen comprising:
(a) in the AM, 20 mg beta adrenergic receptor antagonist (a beta blocker), e.g., a propranolol or equivalent, 200 mg NSAID, e.g., an etodolac or equivalent; in the afternoon, 10 mg beta blocker, 200 mg NSAID, e.g., an etodolac or equivalent; in the PM, 10 mg beta blocker, 400 mg NSAID;
(b) in the AM 40 mg beta adrenergic receptor antagonist (a beta blocker), e.g., a propranolol or equivalent, 200 mg NSAID, e.g., an etodolac or equivalent; in the afternoon 20 mg beta blocker, 200 mg NSAID; in the evening, 20 mg propranolol, 400 mg NSAID;
(c) in the AM 80 mg beta adrenergic receptor antagonist (a beta blocker), e.g., a propranolol or equivalent, 200 mg NSAID; in the afternoon 40 mg beta blocker, 200 mg NSAID, in the evening 40 mg, NSAID; or
(d) a dose escalation comprising a regimen of (a) to (b) to (c).

23. The therapeutic combination of claim 22, wherein:
(a) the beta adrenergic receptor antagonist or beta blocker or equivalent or propranolol or equivalent; the non-steroidal anti-inflammatory drug or NSAID or equivalent or etodolac or equivalent; and the therapeutic agent for the treatment of cancer, are packaged in dosages that match a chrono-dosing regimen comprising:
Start: AM, 20 mg propranolol, 200 mg etodolac; afternoon, 10 mg propranolol, 200 mg etodolac; PM 5 mg propranolol, 400 mg etodolac;
Dose Escalation 1: AM 40 mg propranolol, 200 mg etodolac; afternoon 20 mg propranolol, 200 mg etodolac; evening, 10 mg propranolol, 400 mg etodolac;
Dose escalation 2: AM 80 mg propranolol, 200 mg etodolac; afternoon 40 mg propranolol, 200 mg etodolac, evening 20 mg, etodolac
(b) the therapeutic drug combination is formulated for administration once a day, b.i.d. or t.i.d, or weekly, or biweekly, or monthly; or
(c) the beta adrenergic receptor antagonist (a beta blocker) or propranolol or equivalent; the non-steroidal anti-inflammatory drug or NSAID or etodolac or equivalent; and the therapeutic agent for the treatment of cancer, are formulated for administration once a day, b.i.d. or t.i.d, or weekly, or biweekly, or monthly.

24-26. (canceled)

27. A device, a medical device, an implant, a breast implant, a prosthesis, a stent, a catheter, comprising a therapeutic combination of therapeutic agents or drugs as set forth in claim 1.

28. A pharmaceutical composition or formulation comprising:
(a) the therapeutic combination of claim 1;
(b) the pharmaceutical composition or formulation of (a), further comprising a pharmaceutically acceptable excipient; or
(c) the pharmaceutical composition or formulation of (a) or (b), wherein the pharmaceutical composition or formulation is formulated or manufactured as a feed, a food, a food or feed concentrate, a pellet, a lozenge, a liquid, a lotion, an implant, a nanoparticle, an elixir, an aerosol, a spray, an aerosol, an inhalant, a powder, a tablet, a pill, a capsule, a gel, a geltab, a nanosuspension, a nanoparticle, a patch, a microgel or a suppository.

29-30. (canceled)

31. A method for treating, preventing or ameliorating a tumor or a cancer, comprising:
(a) (i) applying or administering to an individual in need thereof; or, applying or administering to an effected tissue: the pharmaceutical composition or formulation of claim 28,
wherein optionally the therapeutic agents or drugs are administered separately or together, or at the same time, or in synchrony, or by chrono-dosing, or one of the therapeutic agents or drugs is administered before another of the therapeutic agents or drugs,
and optionally the therapeutic agents or drugs are formulated for administration intravenously (IV), parenterally, nasally, topically or locally, orally, or by liposome, implant or vessel-targeted nanoparticle delivery or
(ii) the method of (i), wherein the cancer or tumor is: a mastocytoma or a mast cell tumor, an ovarian cancer, pancreatic cancer, a non-small cell lung cancer, small cell lung cancer, hepatocarcinoma, melanoma, retinoblastoma, breast tumor, colorectal carcinoma, leukemia, lymphoma, acute lymphoblastic leukemia (ALL) or acute lymphoid leukemia, acute myeloid leukemia (AML), a histiocytic sarcoma, a brain tumor, an astrocytoma, a glioblastoma, a neuroma, a neuroblastoma, a colon carcinoma, cervical carcinoma, sarcoma, prostate tumor, bladder tumor, tumor of the reticuloendothelial tissues, Wilm's tumor, ovarian carcinoma, a bone cancer, an osteosarcoma, a renal cancer, or head and neck cancer, oral cancer, a laryngeal cancer, or an oropharyngeal cancer; or
(b) the method of (a), further comprising: an anti-cancer or anti-tumor radiotherapy or a proton beam therapy.

32. (canceled)

33. A method for treating, preventing or ameliorating a tumor or a cancer, comprising:
(a) (i) applying or administering to an individual in need thereof; or, applying or administering to an effected tissue; the pharmaceutical composition or formulation of claim 28,
wherein optionally the therapeutic agents or drugs are administered separately or together, or at the same time, or in synchrony, or by chrono-dosing, or one of the therapeutic agents or drugs is administered before another of the therapeutic agents or drugs,
and optionally the therapeutic agents or drugs are formulated for administration intravenously (IV), parenterally, nasally, topically or locally, orally, or by liposome, implant or vessel-targeted nanoparticle delivery; and
(ii) administering to the individual in need thereof:
(i) a systemic anti-cancer or anti-tumor treatment, wherein optionally the systemic anti-cancer or anti-tumor treatment comprises administration of a drug, a biologic, a nutrient, an anti-cancer or anti-tumor dietary regimen, a radioactive agent, a tumor ablative agent, or
(ii) an anti-cancer or anti-tumor radiotherapy or a proton beam therapy,
wherein the therapeutic combination or pharmaceutical composition or formulation of (a) is administered before the anti-cancer or anti-tumor treatment of (b), or both are administered consecutively, or the therapeutic combination or pharmaceutical composition or formulation of (a) is administered after the anti-cancer or anti-tumor treatment of (b), or any combination thereof; or
(b) the method of (a), further comprising: an anti-cancer or anti-tumor radiotherapy or a proton beam therapy.

34-37. (canceled)
